(19) United States
(12) Patent Application Publication
(10) Pub. No.: US 2017/0299547 A1
(43) Pub. Date: Oct. 19, 2017

(54) CAPILLARY ELECTROPHORESIS APPARATUS

(71) Applicant: SHIMADZU CORPORATION, Kyoto-shi (JP)

(72) Inventors: Akihiro ARAI, Kyoto-shi (JP); Taigo NISHIDA, Kyoto-shi (JP)

(73) Assignee: SHIMADZU CORPORATION, Kyoto-shi (JP)

(21) Appl. No.: 15/131,188

(22) Filed: Apr. 18, 2016

(51) Int. Cl. G01N 27/447; C12Q 1/68

(52) U.S. Cl. CPC G01N 27/44791; C12Q 1/6806; G01N 27/44743

(57) ABSTRACT

A pump is used to draw in a sample into a sample suction tube, and the sample is introduced into a capillary tube that is disposed straight in the horizontal direction. Because the sample is drawn in through the sample suction tube in this way, there is no need to bend the capillary tube so as to dip an end portion thereof into the sample. The result is that there is no need to lengthen the capillary tube to compensate for the degree of separation that is lost through bending of the capillary tube, and thus the capillary tube may be shorter, enabling the analysis to be performed more quickly and with higher throughput.

### TECHNICAL FIELD

The present invention relates to a capillary electrophoresis apparatus for introducing a sample into a capillary tube and performing electrophoresis.

### BACKGROUND ART

Capillary electrophoresis (CE), as a method for rapidly isolating and analyzing minute samples of organisms, with high efficiency and high degree of separation (or resolution), has become an indispensable technology in a broad range of fields of application, such as analysis of molecular structures, such as those of glycoproteins, antibody preparation, low-molecular ionic components, and the like, in addition to the field of genetic analysis, including DNA analysis and sequence analysis using the Sanger technique. There have been proposals for a variety of capillary electrophoresis apparatuses as apparatuses for carrying out analyses using such capillary electrophoresis (referencing, for example, Patent Document 1, listed below).

FIG. 9 is a schematic diagram illustrating an example configuration for a conventional capillary electrophoresis apparatus. This capillary electrophoresis apparatus comprises, in addition to a plurality of capillary tubes 101 (for example, eight capillary tubes): a detector 102, a temperature controlling portion 103, a polymer bottle 104, a polymer filling pump 105, a check valve 106, a buffer valve 107, and anode-side reservoir 108, a cathode-side reservoir 109, an anode 110, a cathode 111, a high-voltage power supply 112, a sample injection cathode 113, a syringe 114, a waste fluid bottle 115, an auto-sampler 116, a controlling portion 117, and the like.

One end of each capillary tube 101 structures a sample injecting end 101a for injecting a sample. Additionally, the other end of each capillary tube 101 structures a joining end 101b, through being joined together. The detector 102 is provided at the joining end 101b side of a plurality of capillary tubes 101, where the length from the sample injecting end 101a to a detecting window (not shown) that is formed on the capillary tube 101 within the detector 102 (the effective length of separation) is, for example, 50 cm.

The plurality of capillary tube 101 is contained in a temperature controlling portion 103, in a state wherein the parts aside from those in the vicinity of the sample injecting end 101a and the vicinity of the joining end 101b are bent. This enables the sample to be subjected to electrophoresis within the capillary tube 101 while the capillary tube 101 is heated during the analysis.

The temperature within the temperature controlling portion 103 is a critical parameter in relation to controlling the degree of separation, electrophoretic velocity (or mobility), and state of mutation of a single DNA strand. Because of this, the temperature controlling portion 103 is structured so as to enable high-accuracy temperature control in a range from room temperature to about 70° C., using a method such as, for example, an oven with circulating air or a method wherein the capillary tube 101 is brought into contact with a surface for which the temperature is controlled.

A separating polymer is contained in the polymer bottle 104. One end portion of a polymer suction tube 121 is connected to a suction opening of the polymer filling pump 105, and the other end portion of the polymer suction tube 121 is inserted into the polymer bottle 104. A check valve 106 is installed in the polymer suction tube 121, to enable prevention of reverse flow of the separating polymer from the polymer filling pump 105 side into the polymer bottle 104.

One end portion of a polymer supply tube 122 is connected to the discharge outlet of the polymer filling pump 105, and the other end portion of the polymer supply tube 122 is connected to the joining end 101b of a capillary tube 101. Moreover, one end portion of an anode-side connecting tube 123 is connected to the joining end 101b of a capillary tube 101, and the other end portion of the anode-side connecting tube 123 is inserted into an anode-side reservoir 108 that holds a buffer solution. A buffer valve 107 is installed in the anode-side connecting tube 123. The anode 110 is arranged in the anode-side reservoir 108 in a state wherein it is immersed in the buffer solution.

In this capillary electrophoresis apparatus, prior to carrying out an analysis, a separating polymer is filled into each capillary tube 101, the polymer supply tube 122, and the anode-side connecting tube 123. Specifically, first the polymer filling pump 105 is driven to draw in the separating polymer from the polymer bottle 104. In this case, the buffer valve 107 is placed into the open state, so that the entire length of the polymer suction tube 121, the polymer supply tube 122, and the anode-side connecting tube 123 will be filled with the separating polymer.

Once the separating polymer has been drawn out by the polymer filling pump 105, it will not then flow back into the polymer bottle 104 due to the check valve 106, and there will be essentially no fill into the individual capillary tubes 101 either, due to the back pressure. The result is that the separating polymer that has been drawn in is expelled into the anode-side reservoir 108.

Following this, the polymer filling pump 105 is driven again, and the separating polymer is drawn in from the polymer bottle 104. In this case, the buffer valve 107 is in a closed state, and so each of the capillary tubes 101 is refilled with separating polymer. The separating polymer that is expelled for from the sample injecting end 101a of each of the capillary tubes 101 flows into a cathode-side reservoir 109, which holds the buffer solution. In the cathode-side reservoir 109, a cathode 111 is disposed in a state wherein it is immersed in the buffer solution.

Typically, the separating polymer includes a high viscosity polymer matrix and a DNA modifying agent. Because of this, these must be replaced periodically, after trapping moisture on the seal member (not shown), so that there will be no solidification or deposition within the polymer filling pump 105. Such an operation may be carried out through a manual operation using a syringe 114 and a waste fluid bottle 115 that are each connected to the polymer filling pump 105.

When injecting samples into each of the capillary tubes 101, the sample injecting ends 101a of each of the capillary tubes 101 are inserted into a plurality of sample containers 130 through the use of the auto-sampler 116. A sample injection cathode 113 is provided at the sample injecting end 101a of each capillary tube 101. In a state wherein the sample injection cathode 113 of each individual capillary tube 101 is immersed in a sample within the sample container 130, a high-voltage power supply 112 is used to apply a voltage between the sample injection cathode 113 and the anode 110 within the anode-side reservoir 108, to inject the samples into the individual capillary tubes 101.

At the time of sample injection, the voltage that is applied by the high-voltage power supply 112, and the time over which the voltage is applied, are controlled by a controlling portion 117. When the injection of the samples into each of the capillary tubes 101 has been completed, then the auto-sampler 116 operates so as to immerse the sample injecting ends 101a of each of the capillary tubes 101 into the buffer solution within the cathode-side reservoir 109. In this state, a voltage is applied, using the high-voltage power supply 112, between the cathode 111 within the cathode-side reservoir 109 and the anode 110 within the anode-side reservoir 108, to start electrophoretic separation of the sample.

The separated DNA fragments pass through the detector 102 following the order of the length of the strand, and are expelled into the buffer solution within the anode-side reservoir 108. The detector 102 is provided with excitation beam optics for emitting an excitation beam of a constant and uniform strength into each of the capillary tubes 101, and a spectral optical axial direction system for spectrally separating the fluorescence produced from each of the capillary tubes 101. As the source for the stimulating beam, a laser diode (LD), or an LD-stimulated solid-state laser may be used. A reflective or transmissive diffraction grating, a prism, or the like, may be used as the spectral separating means in the spectral optical system. A backface-incident CCD area image sensor, an area scan CMOS image sensor, or the like, may be used as the photodetecting element.

### PRIOR ART DOCUMENTS

### Patent Document

[Patent Document 1] U.S. Pat. No. 6,531,044

### SUMMARY OF THE INVENTION

### Problem Solved by the Present Invention

In recent years, there has been active work in comprehensive analysis of full genomes, using next-generation sequencers (NGS) in order to search for target genetic mutations for new molecule-targeted drugs, to search for disease-related gene markers, and to evaluate safety looking toward clinical applications of iPS cells, and so forth. As part of this, sequencing using the Sanger method, using capillary electrophoresis apparatuses, has been considered as means for complementing NGS and for isolation for routine examinations of individual genes.

For example, in analyses of cancer and known genetic mutations of congenital illnesses, or in systematic analysis of microorganisms and pathogens, there is the need to perform high-throughput analysis more rapidly for relatively short DNA base lengths. Moreover, in routine examination in industry, there is the need for operations and maintenance to be simple, in addition to processing performance.

In such a flow, in a conventional capillary electrophoresis apparatus, as described above, the effective length of separation is, for example, 50 cm, and when carrying out electrophoresis under standard conditions using the commercially available POP7 (manufactured by Applied Biosystems) as the separating polymer, on average it is possible to sequence 850 bases in 125 minutes. Additionally, in high-speed mode it is possible to sequence 500 bases in about 40 minutes. However, even in such a case, analysis of about 280 samples per day using eight capillary tubes is the upper limit, and thus it would be desirable to have a capillary electrophoresis apparatus able to perform analyses at higher speeds with higher throughput.

The present invention was created in contemplation of the situation set forth above, and the object is to provide a capillary electrophoresis apparatus able to enable analyses at higher speeds with higher throughput.

### Means for Solving the Problem

The capillary electrophoresis apparatus according to the present invention comprises: a sample suction tube for drawing in a sample; a pump that is driven in order to draw in a sample into the sample suction tube; and a capillary tube, disposed straight in the horizontal direction, into which the sample that is drawn into the sample suction tube is introduced.

Such a structure enables the sample to be drawn into a sample suction tube using a pump, and introduced into a capillary tube that is disposed straight in the horizontal direction. Because the sample is drawn in through a sample suction tube in this way, there is no need to bend the capillary tube to dip an end portion in the sample. As a result, there is no need to lengthen the capillary tube in order to make up for the degree of separation (or resolution) that is lost through bending of the capillary tube, making it possible for the capillary tube to be shorter, thereby enabling the analysis to be faster with a higher throughput. Moreover, because there is no need to immerse the electrode in the sample container each time electrical injection is performed, the electrodes do not become contaminated with the sample, and there are no concerns that the sample may remain between the electrode and the capillary tube.

The capillary electrophoresis apparatus may further comprise: a cathode-side block wherein a cathode-side end portion of the capillary tube, the sample suction tube, and the pump are connected, and wherein an interior space is formed wherein the flows thereof are joined.

With this structure, it is possible to draw in, into the space within a cathode-side block, the sample that has been drawn in to the sample suction tube using the pump, to introduce the sample into the cathode-side end portion of the capillary tube from this interior space. This enables the sample to be introduced into the capillary tube reliably and with stability, through the cathode-side block.

A plurality of capillary tubes and a plurality of the sample suction tubes, corresponding to the individual capillary tubes, may be connected to the cathode-side block, and a plurality of interior spaces, corresponding to the individual capillary tubes, may be formed in the cathode-side block.

This structure enables samples to be drawn in simultaneously into a plurality of sample suction tubes, where after the samples have been drawn into the respective individual interior spaces within the cathode-side block, the samples can be introduced into the cathode-side end portions of the plurality of capillary tubes that correspond to the interior spaces. This enables the samples to be introduced simultaneously into the respective plurality of short capillary tubes, enabling the analysis to be carried out more quickly, with higher throughput.

The capillary electrophoresis apparatus may further comprise: a cathode-side reservoir having a buffer solution retaining portion able to hold a buffer solution; and a cathode that is immersed in the buffer solution in the cathode-side reservoir.

This structure enables the introduction of samples into the cathode-side end portions of the capillary tubes through merely applying a voltage to the cathode within the cathode-side reservoir through dipping the sample suction tube into the buffer solution within the cathode-side reservoir after drawing samples into the interior spaces of the cathode-side block through the sample suction tube.

The capillary electrophoresis apparatus may further comprise: a cathode-side reservoir that has a buffer solution retaining portion able to hold a buffer solution, where the buffer solution retaining portion is connected to an interior space of the cathode-side block; and a cathode that is immersed in the buffer solution in the cathode-side reservoir. In this case, when a sample is drawn into an interior space of the cathode-side block through the sample suction tube, a voltage may be applied to the cathode within the cathode-side reservoir, after buffer solution within the cathode-side reservoir has also been drawn in, to introduce a sample into the capillary tube from the cathode-side end portion.

This structure enables the buffer solution within the cathode-side reservoir to be drawn in as well when drawing the samples into the interior spaces within the cathode-side block through the sample suction tubes. The application of a voltage to the cathode within the cathode-side reservoir thereafter enables the samples to be introduced into the cathode-side end portions of the capillary tubes, which can shorten the time when introducing the samples, thus enabling the analyses to be performed at higher speeds with higher throughput. Moreover, if the width of the flow path for the sample suction flow path is sufficiently small in relation to the flow path of the buffer communicating flow path, then the samples of the capillary ends can be introduced completely, which can improve quantifiability.

The capillary electrophoresis apparatus may further comprise: an anode-side reservoir block able to hold a buffer solution within a buffer solution retaining portion, and wherein an interior space is formed connecting with an anode-side end portion of the capillary tube; and a polymer filling mechanism for pressure-filling a separating polymer through the interior space of the anode-side reservoir block into the capillary tube from the anode-side end portion.

This structure enables filling of the separating polymer into the capillary tubes from the anode-side end portions through the interior spaces of the anode-side reservoir block. Because the buffer solution of the anode-side reservoir block and the separating polymer make contact after filling of the separating polymer, this enables electrophoresis to be carried out without switching a valve in the anode-side reservoir.

The polymer filling mechanism may include a polymer filling needle that is inserted into the anode-side reservoir block. In this case, a connecting port, into which the tip end of the polymer filling needle can be inserted, and which can be sealed thereby, may be formed at a boundary portion, in the anode-side reservoir block, between the buffer solution retaining portion and the interior space.

This structure enables the tip end of the polymer filling needle to be inserted into a connecting port of the anode-side reservoir block, to enable the separating polymer to be filled into the capillary tubes through the interior spaces of the anode-side reservoir block. This enables easy forcing of the separating polymer into the capillary tubes without having to carry out an operation to eliminate bubbles.

The capillary electrophoresis apparatus may further comprise: a rinsing water supplying mechanism for supplying rinsing water through the interior space of the anode-side reservoir block to the capillary tube from the anode-side end portion.

This structure enables easy cleaning of the interior of the capillary tubes through merely supplying rinsing water from the anode-side end portions to the capillary tubes through the interior spaces of the anode-side reservoir blocks. Moreover, this also enables the rinsing water to clean the interior spaces of the anode-side reservoir block, and to also clean the buffer solution retaining portions that communicate with the interior spaces, and enables a state wherein, after cleaning, the anode-side end portions of the capillary tubes are in contact with the rinsing water, making it possible to prevent the anode-side end portions from drying out. Maintainability can be improved through carrying out such procedures automatically.

The rinsing water supplying mechanism may include a rinsing water supplying needle wherein the tip end thereof can be inserted into the connecting port.

This structure enables the tip ends of the rinsing water supplying needles to be inserted into the connecting ports of the anode-side reservoir block, enabling the rinsing water to be supplied to the capillary tubes through the interior spaces of the anode-side reservoir block from the tip ends of the rinsing water supplying needles. This enables easy forcing of the rinsing water into the capillary tubes.

The capillary electrophoresis apparatus may further comprise: a temperature controlling portion, provided straight along the direction in which the capillary tube extends, able to contain, and control the temperature of, the capillary tube therein. In this case, the capillary tube may be removed to the outside of the temperature controlling portion through moving the temperature controlling portion in a direction that is perpendicular to the direction in which the capillary tube extends.

This structure enables the temperature to be controlled uniformly over essentially the entire region, except for the fittings on both ends of the capillary tubes, through the temperature controlling portion wherein the capillary tubes that are arranged straight in the horizontal direction being provided straight along that same direction. Moreover, merely moving the temperature controlling portion in the direction that is perpendicular to the direction in which the capillary tubes extend enables easy removal to the outside of the temperature controlling portion, enabling an improvement in maintainability.

### Effects of the Invention

The present invention enables capillary tubes to be short, because there is no need to lengthen the capillary tubes in order to compensate for the degree of separation that is lost due to bending of the capillary tubes, thus enabling analyses to be carried out at higher speeds with higher throughput. Moreover, because the capillary tubes are not inserted directly into the sample containers, the electrodes do not contaminate the samples. Because the temperature is controlled uniformly over the entire region of the capillary tubes, with the exception of the connecting portions on both ends, it is possible to produce maximum degree of separation, even with short capillary tubes. Moreover, after an analysis has been completed, the interiors of the capillary tubes can be cleaned easily with water, and thus there is superior ease of operation and superior maintainability

### BRIEF DESCRIPTION OF THE DRAWINGS

FIG. 1 is a perspective diagram illustrating an example configuration of a capillary electrophoresis apparatus according to an embodiment according to the present invention.

FIG. 2 is a schematic drawing of a capillary electrophoresis apparatus of FIG. 1.

FIG. 3 is a plan view illustrating an example configuration of a capillary bundle.

FIG. 4A is a cross-sectional view illustrating a form wherein the polymer filling needles can be inserted removably into the connecting ports on the anode-side reservoir block, illustrating the state wherein the polymer filling needles are connected to the connecting ports.

FIG. 4B is a cross-sectional view illustrating a form wherein the polymer filling needles can be connected removably into the connecting ports on the anode-side reservoir block, illustrating a state wherein the connecting ports are open.

FIG. 5 is a perspective diagram illustrating a form wherein samples are introduced into each of the individual capillary tubes.

FIG. 6 is a diagram illustrating an example configuration of a detecting portion.

FIG. 7 is a schematic diagram illustrating an example configuration of a capillary electrophoresis apparatus according to another embodiment according to the present invention.

FIG. 8 is a perspective diagram illustrating a form wherein samples are introduced into each of the individual capillary tubes in the capillary electrophoresis apparatus of FIG. 7.

FIG. 9 is a schematic diagram illustrating an example configuration of a conventional capillary electrophoresis apparatus.

### DETAILED DESCRIPTION OF THE EXEMPLARY EMBODIMENTS

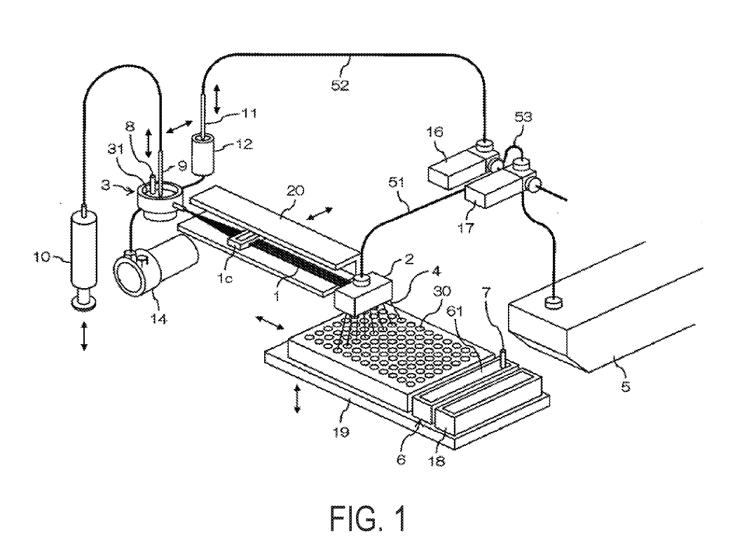

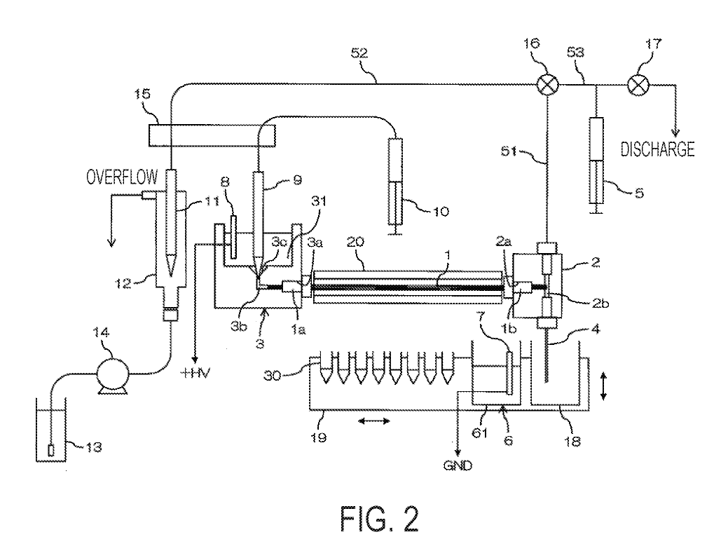

FIG. 1 is a perspective diagram illustrating an example configuration of a capillary electrophoresis apparatus according to an embodiment according to the present invention. Moreover, FIG. 2 is a schematic drawing of the capillary electrophoresis apparatus of FIG. 1.

This capillary electrophoresis apparatus comprises, in addition to a plurality (for example, eight) capillary tubes 1: a cathode-side block 2, an anode-side reservoir block 3, sample suction tubes 4, a pump 5, a cathode-side reservoir 6, a cathode 7, an anode 8, a polymer filling needle 9, a polymer cartridge 10, a rinsing water supplying needle 11, a rinsing port 12, a rinsing water tank 13, a rinsing water pump 14, a driving mechanism 15, an electromagnetic switching valve 16, an opening/closing valve 17, a drain 18, an auto-sampler 19, a temperature controlling portion 20, and the like.

A plurality of capillary tubes 1 is disposed straight in the horizontal direction, in a grouped state. One end portion (the cathode-side end portion) of each capillary tube 1 is connected to the cathode-side block 2, where the other end portion (the anode-side end portion) is connected to the anode-side reservoir block 3. A sample suction tube 4 is provided for each capillary tube 1, and the respective end portions thereof are connected to the cathode-side block 2. Moreover, a pump 5 is also connected, through a pipe 51, to the cathode-side block 2.

Each individual sample suction tube 4 is to draw in a sample, and a pump 5 that is connected through the cathode-side block 2 and the pipe 51 is connected to the sample suction tubes 4, to enable samples to be drawn into each of the individual sample suction tubes 4. The sample that is drawn into a sample suction tube 4 is introduced into the respective capillary tube 1 from the cathode-side end portion of the individual capillary tube 1 that is connected through the cathode-side block 2.

The cathode-side reservoir 6 has a buffer solution retaining portion 61 that is able to hold a buffer solution, where a cathode 7 is disposed, in an immersed state, in the buffer solution within the buffer solution retaining portion 61. Moreover, the anode-side reservoir block 3 has a buffer solution retaining portion 31 that holds a buffer solution, where an anode 8 is disposed, in an immersed state, in the buffer solution within the buffer solution retaining portion 31. The cathode-side reservoir 6 and the anode-side reservoir block 3 are each formed from an insulating material, such as, for example, a resin.

The polymer filling needle 9 is connected to the polymer cartridge 10. In a state wherein the polymer filling needle 9 is inserted into the anode-side reservoir block 3, the separating polymer within the polymer cartridge 10 is supplied into the polymer filling needle 9, and the separating polymer is filled into the capillary tube 1 through the anode-side reservoir block 3 from the tip end of the polymer filling needle 9. The polymer filling needle 9 and the polymer cartridge 10 structure a polymer filling mechanism for pressurized filling of the separating polymer from the anode-side end portion into the capillary tube 1.

The rinsing water supplying needle 11 is connected to the pump 5 through the pipe 52. Rinsing water that is contained within the rinsing water tank 13 is supplied through the rinsing water pump 14 to a rinsing port 12. In a state wherein the rinsing water supplying needle 11 is inserted into the rinsing port 12, the pump 5 is driven to enable the rinsing water to be drawn into the rinsing water supplying needle 11 and the interior of the pipe 52.

Thereafter, the rinsing water supplying needle 11, when in a state wherein it is inserted into the anode-side reservoir block 3, is driven by the pump 5 to supply the rinsing water, within the rinsing water supplying needle 11 and the pipe 52, into the interior of the capillary tubes 1 through the anode-side reservoir block 3 from the tip end of the rinsing water supplying needle 11. The rinsing water supplying needle 11, the rinsing port 12, the rinsing water tank 13, and the rinsing water pump 14 structure a rinsing water supplying mechanism for supplying rinsing water from the anode-side end portions into the capillary tubes 1.

The driving mechanism 15 is driven when moving the polymer filling needle 9 or the rinsing water supplying needle 11. This driving mechanism 15 enables either the polymer filling needle 9 or the rinsing water supplying needle 11 to be inserted into the anode-side reservoir block 3 or inserted into the rinsing port 12.

The pipes 51 and 52 are connected together through an electromagnetic switching valve 16, to be connected to the pump 5 through a shared pipe 53. This electromagnetic switching valve 16 can be switched to enable switching between a state wherein the pump 5 is connected through the pipe 51 to the cathode-side block 2, and a state wherein the pump 5 is connected to the rinsing water supplying needle 11 through the pipe 52. An opening/closing valve 17 is provided in the pipe 53.

The drain 18 is for holding waste fluid, where, in this example, it is provided lined up with the cathode-side reservoir 6. The cathode-side reservoir 6 and the drain 18 are held on the auto-sampler 19 together with a plurality of sample containers 30. Consequently, the auto-sampler 19, through being moved in the horizontal direction and the vertical direction, enables the sample suction tubes 4 to be inserted into the sample containers 30, the cathode-side reservoir 6, or the drain 18.

The temperature controlling portion 20 is provided straight along the direction in which the capillary tubes 1 extend. In this example, the temperature controlling portion 20 is formed with a cross-sectional shape that is a U, open on one side face thereof, where the temperature controlling portion is covered by a thermal insulation material for actual operation, and through moving the temperature controlling portion 20 in the horizontal direction that is perpendicular to the direction in which the capillary tubes 1 extend, the capillary tubes 1 may be contained within the temperature controlling portion 20, or the capillary tubes 1 may be removed to the outside of the temperature controlling portion 20 through the one open side face.

Note that the temperature controlling portion 20 need not be limited to a shape wherein the cross-suction is a U shape, insofar as it is a shape that can contain the capillary tubes 1 therein, but may be a different shape instead. Moreover, the direction in which the temperature controlling portion 20 is moved is not limited to a horizontal direction that is perpendicular to the direction in which the capillary tubes 1 extend, but rather may be another direction instead that is perpendicular to the direction in which the capillary tubes 1 extend.

Specific structures and operations of the capillary electrophoresis apparatus according to the present embodiment will be explained in detail below, referencing FIG. 1, FIG. 2, and, as appropriate, other drawings. The operation of the capillary electrophoresis apparatus is controlled by a controlling portion (not shown) that includes, for example, a CPU (a Central Processing Unit), where operations such as explained below can be carried out automatically through the execution of a program by the CPU.

### <Attachment of the Capillary Group, Temperature Control, and Rinsing>

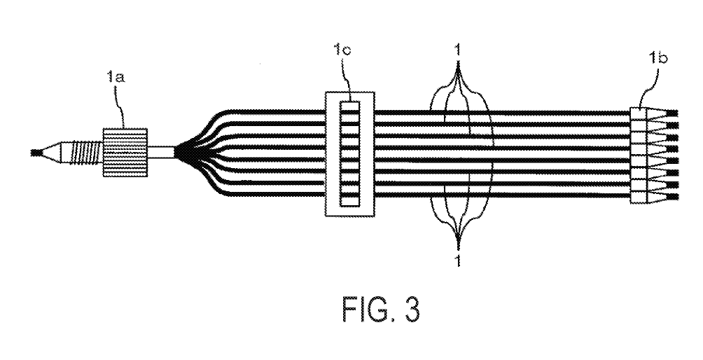

FIG. 3 is a plan view illustrating an example configuration of a capillary group. In the present embodiment, a plurality (for example, eight) capillary tubes 1 is joined together at the anode-side end portions, to structure a capillary group. The individual capillary tubes 1 are disposed in straight lines, so as to be parallel to each other, with prescribed spacing therebetween.

The plurality of capillary tubes 1 are joined on the anode-side end portions thereof, and attached to the male nuts 1a, manufactured from, for example, PEEK. This male nut 1a structures an anode-side attaching portion for attaching the anode-side end portion of the capillary tube 1 to the anode-side reservoir block 3. As illustrated in FIG. 2, an attaching hole 3a, for attaching the male nut 1a, is formed in the side face of the anode-side reservoir block 3, enabling the male nut 1a to be screwed into the attaching hole 3a.

Fittings 1b made from, for example, PEEK, are attached to the cathode-side end portions of the plurality of capillary tubes 1. The fittings 1b that are attached to the cathode-side end portions of the capillary tubes 1 are connected together, to structure, in the present example, eight connected fittings. The tip end portion of each individual fitting 1b is formed from a conical tapered surface. As illustrated in FIG. 2, a plurality of attaching holes 2a, for attaching the individual fittings 1b, are formed in a side face of the cathode-side block 2, where the individual fittings 1b can be attached through pressing into the attaching holes 2a.

The anode-side end portion of each individual capillary tube 1 is secured to a male nut 1a in a state wherein it protrudes slightly beyond the male nut 1a. Similarly, the cathode-side end portion of each capillary tube 1 is secured to the respective fitting 1b in a state wherein it protrudes slightly further than the fitting 1b. The amount by which the anode-side end portion of the individual capillary tube 1 protrudes from the male nut 1a is preferably the same for each, and the amounts by which the cathode end portions of the individual capillary tubes 1 extend beyond the individual fittings 1b are preferably the same for each.

A detecting window 1c, to enable incidence of the light at the time of detection, is provided further toward the anode-side than the center in each of the individual capillary tubes 1. The length (the effective length of separation) from the cathode-side end portion to the detecting window 1c for each individual capillary tube 1 is, for example, no more than 10 cm. In this example, the inner diameter of each individual capillary tube 1 is set to 50 μm, with the effective length of separation set to 85 mm. However, the shape of the individual capillary tube 1 is not limited to this shape. There is no limitation to there being eight capillary tubes 1, but rather there may be seven or fewer, or nine or more, or may be only a single tube rather than a plurality thereof.

As illustrated in FIG. 2, interior spaces 3b that connect between the buffer solution retaining portion 31 and the anode-side end portion of each of the individual capillary tubes 1 are formed in the anode-side reservoir block 3. These interior spaces 3b are formed with a big L-shaped cross-suction, where one end portion is connected to the attaching hole 3a, and the other end portion (the top end portion) is connected to the bottom of the buffer solution retaining portion 31. A connecting port 3c that is, for example, cone-shaped, is formed at the boundary portion between the buffer solution retaining portion 31 and the interior space 3b, at the bottom of the buffer solution retaining portion 31. The tip end of the polymer filling needle 9 and the tip end of the rinsing water supplying needle 11 can be inserted into (pressed into) the connecting port 3c.

Moreover, interior spaces 2b are formed in the cathode-side block 2 at the place wherein the flows of the cathode-side end portions of the individual capillary tubes 1 and the individual samples suction tubes 4 and the pump 5 (the pipe 51) are joined together. An interior space 2b is provided corresponding to each of the plurality of capillary tubes 1, where each interior space 2b is formed extending in the vertical direction, with the bottom end portion thereof connecting to the respective sample suction tube 4, and the top end portions connected in confluence with the pipe 51. The attaching hole 2a for each capillary tube 1 is in communication part way through with each individual interior space 2b that extends in the vertical direction, where the cathode-side end portion of each individual capillary tube 1 that protrudes from the tip end of each fitting 1b that is attached to the respective attaching hole 2a forms a state wherein it is pulled out slightly into the respective interior space 2b (referencing FIG. 2).

In each individual capillary tube 1, the part between the male nut 1a and the fitting 1b has the temperature thereof controlled by the temperature controlling portion 20. The temperature controlling portion 20 is provided with a heater (not shown), wherein the heater can be driven to control the temperature of the capillary tubes 1 contained within the temperature controlling portion 20.

In the present embodiment, the temperatures of the capillary tubes 1 that are arranged straight in the horizontal direction can be controlled uniformly, over essentially the entire region thereof, except for the male nut 1a and the fitting 1b at the two ends of the capillary tubes, through the temperature controlling portion 20 that is provided straight along the same direction. Moreover, the capillary tubes 1 can be removed easily to the outside of the temperature controlling portion 20 by merely moving the temperature controlling portion 20 in a direction that is perpendicular to the direction in which the capillary tubes 1 extend, thus improving maintainability.

Each individual capillary tube 1 is rinsed with water using the procedure described below. First the rinsing water pump 14 is driven to supply rinsing water from within the rinsing water tank 13 to the rinsing port 12, where a portion overflows to the outside. Then the rinsing water supplying needle 11 is dipped into the rinsing water within the rinsing port 12 by the driving mechanism 15, and in a state wherein the pipe 52 and the pipe 53 are connected by the electromagnetic switching valve 16, the pump 5 is driven to draw rinsing water into the pump 5 side.

Thereafter, the electromagnetic switching valve 16 is closed and the pump 5 is driven with the opening/closing valve 17 in the open state, to drain the rinsing water to the outside. Such an operation can be repeated to completely eliminate bubbles within the flow path from the rinsing water supplying needle 11 to the pump 5.

Following this, the rinsing water supplying needle 11 is pressed, by the driving mechanism 15, into the connecting port 3c of the anode-side reservoir block 3, and the pump 5 is driven to supply the rinsing water from the anode-side end portion to each of the capillary tubes 1 through the interior spaces 3b of the anode-side reservoir block 3. The rinsing water that is supplied into each of the capillary tubes 1 is guided from the cathode-side end portion through the cathode-side block 2 to each of the sample suction tubes 4, and drained into the drain 18 from each of the sample suction tubes 4.

<Filling Polymer into the Capillary Group>

The separating polymer is filled into each of the capillary tubes 1 through the procedure set forth below. First, the respective buffer solutions are filled into the buffer solution retaining portion 31 of the anode-side reservoir block 3 and the buffer solution retaining portion 61 of the cathode-side reservoir 6. The polymer filling needle 9 is then inserted into the rinsing port 12, to supply the separating polymer to the polymer filling needle 9 from the polymer cartridge 10. Through this, a prescribed amount of the separating polymer is expelled from the tip end of the polymer filling needle 9.

Following this, the polymer filling needle 9 is pressed, by the driving mechanism 15, into the connecting port 3c of the anode-side reservoir block 3, and the separating polymer is supplied to the polymer filling needle 9 from the polymer cartridge 10, to fill the separating polymer into each of the capillary tubes 1 from the anode-side end portion through the interior spaces 3b of the anode-side reservoir block 3. In this case, the separating polymer that flows out from the cathode-side end portion of each capillary tube 1 is directed into the respective sample suction tube 4 through the cathode-side block 2, to be drained into the drain 18 from the respective sample suction tube 4.

After filling the separating polymer into each capillary tube 1 for a prescribed time interval, the polymer filling needle 9 is removed from the connecting port 3c of the anode-side reservoir block 3 by the driving mechanism 15, to open the connecting port 3c. Thereafter, the pump 5 is driven when in a state wherein the pipe 51 and the pipe 53 are connected by the electromagnetic switching valve 48, to draw into the pump 5 side the rinsing water in the drain 18.

This makes it possible to eliminate the separating polymer that remains within the interior space 2b of the cathode-side block 2. However, there is no limitation to such a structure, but rather the structure may be one wherein the rinsing water within the drain 18 is drawn into the pump 5 side by driving the pump 5 simultaneously when each capillary tube 1 is being filled with the separating polymer.

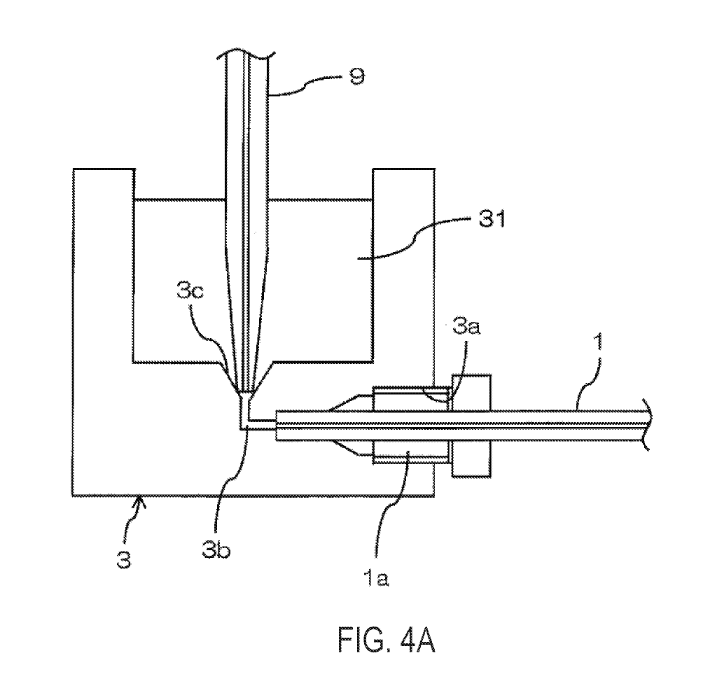

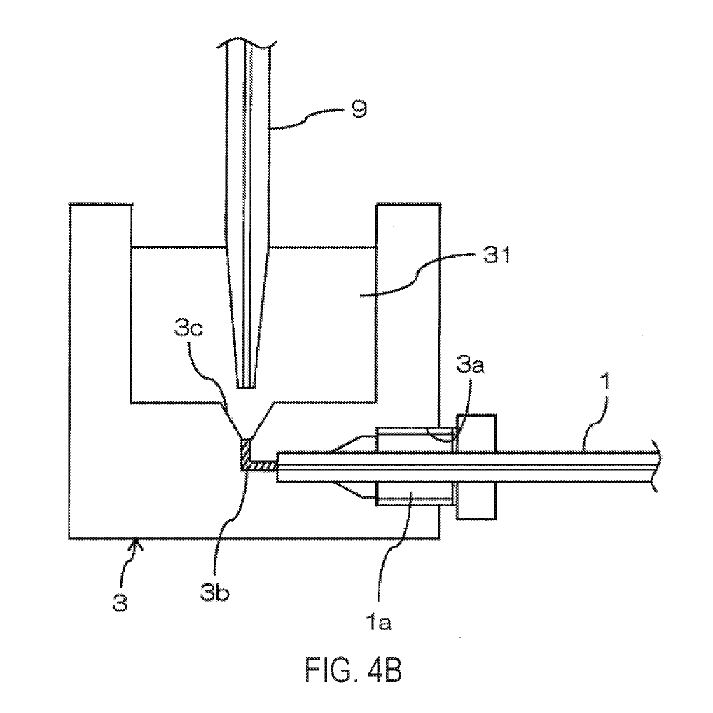

FIG. 4A and FIG. 4B are cross-sectional views illustrating a form wherein the polymer filling needle 9 can be attached removably to the connecting port 3c of the anode-side reservoir block 3, where FIG. 4A shows the state wherein the polymer filling needle 9 is connected to the connecting port 3c, and FIG. 4B shows the state wherein the connecting port 3c is open.

As illustrated in FIG. 4A, if the polymer filling needle 9 is connected to the connecting port 3c, then the state is one wherein the tapered tip end portion of the polymer filling needle 9 is pressed into the similarly tapered connecting port 3c, facing the interior space 3b. Supplying the separating polymer from the polymer filling needle 9 in this state makes it possible to supply the separating polymer to the interior space 3b without flowing into the buffer solution retaining portion 31.

Thereafter, if, as illustrated in FIG. 4B, the polymer filling needle 9 is removed from the connecting port 3c of the anode-side reservoir block 3, to open the connecting port 3c, then, as illustrated by the hatching in the figure, the state will be one wherein the separating polymer remains in the interior spaces 3b.

In the present embodiment, the separating polymer can be filled into the capillary tubes 1 from the anode-side end portions through the interior spaces 3b of the anode-side reservoir block 3. After filling with the separating polymer, the buffer solution of the anode-side reservoir block 3 and the separating polymer are in contact, and thus electrophoresis can be performed in that state.

Moreover, in the present embodiment, the tip end of the polymer filling needle 9 can be inserted into the connecting port 3c of the anode-side reservoir block 3, to fill the separating polymer into the capillary tubes 1 through the interior spaces 3b of the anode-side reservoir block 3 from the tip end of the polymer filling needle 9. This makes it possible to force the separating polymer to flow into the capillary tube 1, without performing an operation to eliminate bubbles.

<Introducing the Samples into the Capillary Group>

The samples are introduced into the individual capillary tubes 1 through the procedure set forth below. First the pipe 51 and the pipe 53 are connected by the electromagnetic switching valve 16, and the drain 18 is moved downward by the auto-sampler 19, to separate the sample suction tubes 4 from the drain 18. The pump 5 is driven when in this state to suck an airspace (for example, 5 μL) into the interior spaces 2b of the cathode-side block 2.

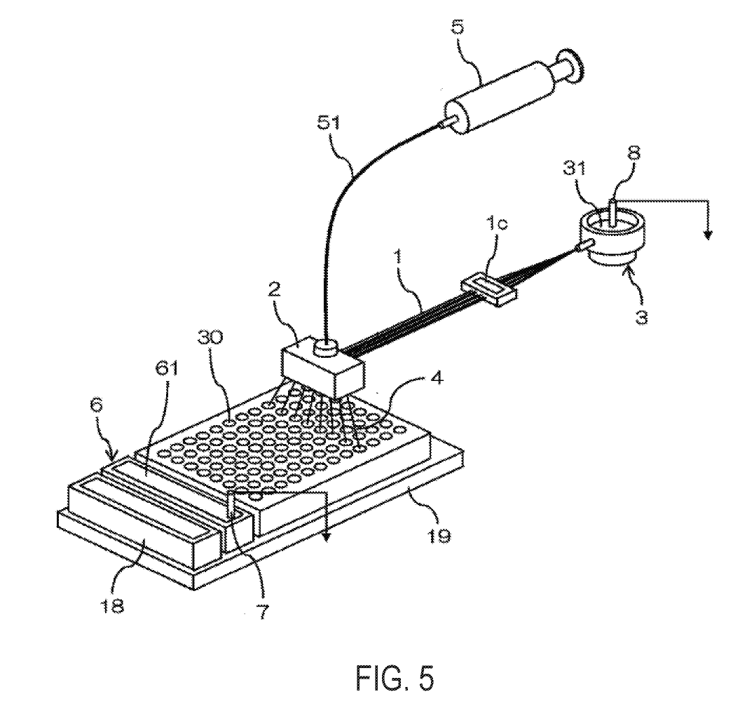

FIG. 5 is a perspective diagram illustrating the form wherein the samples are introduced into the individual capillary tubes 1. After drawing in the air space, the individual sample suction tubes 4 are dipped into the samples within the sample container 30 by the auto-sampler 19, and the pump 5 is driven while in this state, to draw in the prescribed amount of the samples into the interior spaces 2b of the cathode-side block 2 through the individual sample suction tubes 4. In this case, the suction of the samples is, for example, over five seconds at a rate of 1 μL per second, to draw 5 μL samples into the interior spaces 2b of the cathode-side block 2.

Given this, each of the sample suction tubes 4 is dipped into the buffer solution within the cathode-side reservoir 6, by the auto-sampler 19, and a voltage is applied between the cathode 7 and the anode 8, to introduce each sample properly from the cathode-side end portion into the respective capillary tube 1. At this time, the application of the voltage between the cathode 7 and the anode 8 with an electric field strength of 230 V/cm, for example, as the voltage for introducing the samples, enables the samples to be introduced well into the individual capillary tubes 1 from the cathode-side end portions thereof.

After introduction of the samples, the voltage applied between the cathode 7 and the anode 8 is discontinued, and, at the same time, the cathode-side reservoir 6 is moved downward by the auto-sampler 19, to separate the sample suction tubes 4 from the cathode-side reservoir 6. The pump 5 is driven while in this state, to draw in an air space (for example, 10 μL) into the interior spaces 2b of the cathode-side block 2.

Following this, the individual sample suction tubes 4 are dipped into the buffer solution within the cathode-side reservoir 6 by the auto-sampler 19, and the pump 5 is driven to draw in a prescribed amount of the buffer solution. In this case, the buffer solution is drawn in for 10 seconds at a rate of, for example, 1 μL per second, to draw 10 μL of the buffer solution into the interior spaces 2b of the cathode-side block 2.

In the present invention, the samples can be drawn into the sample suction tubes 4 by the pump 5 and introduced into the capillary tubes 1 that are disposed straight in the horizontal direction. In this way, the samples are drawn in through the sample suction tubes 4, so there is no need to bend the capillary tubes 1 to dip the end portions thereof into the samples. The result is that it is not necessary for the capillary tubes 1 to be long enough to compensate for the degree of separation lost through bending the capillary tubes 1, enabling the capillary tubes 1 to be shorter, thus enabling the analysis to be performed more quickly, with a higher throughput.

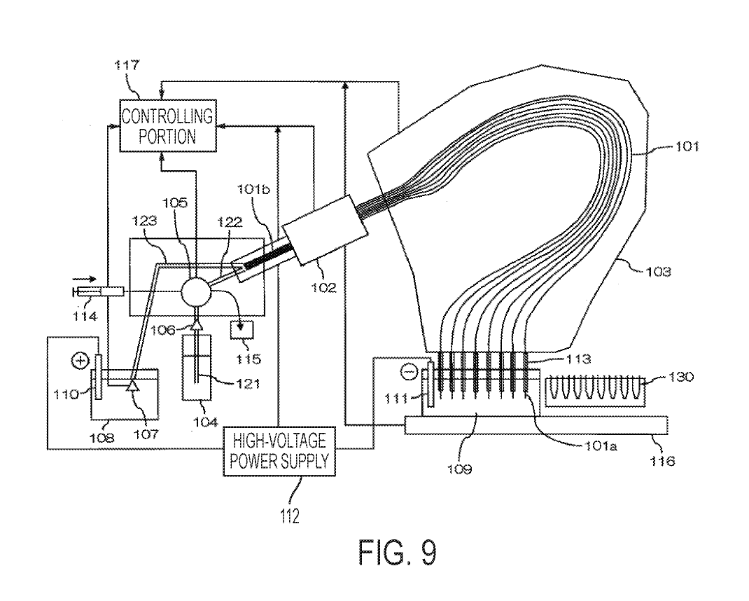

For example, in a case wherein the inner diameter of each capillary tube 1 is 50 μm and the effective length of separation is 85 mm, then, with the capillary electrophoresis apparatus according to the present embodiment, it is possible to sequence a 300-base length in about 10 minutes. In a 15-minute cycle, using eight capillary tubes 1, it is possible to analyze 32 samples in one hour, or to analyze 768 samples in 24 hours, meaning that it is possible to perform analyses with a high throughput when compared to the conventional capillary electrophoresis apparatus illustrated in FIG. 9 (wherein the upper limit for the analyses within one day is about 280 samples).

Moreover, in the present embodiment, the pump 5 can be used to draw in, into the interior spaces 2b of the cathode-side block 2, the samples that have been drawn into the sample suction tubes 4, to introduce the samples into the cathode-side end portions of the capillary tubes 1 from the interior spaces 2b. This makes it possible to introduce the samples into the capillary tubes 1 through the cathode-side block 2 with reliability and stability.

In particular, because a plurality of interior spaces 2b that correspond to the individual capillary tubes 1 is formed within the cathode-side block 2, the samples within the plurality of sample suction tubes 4 can be drawn in simultaneously, and these samples can be introduced into the cathode-side end portions of the plurality of capillary tubes 1, corresponding to the respective interior spaces 2b, after these samples are drawn into the respective individual interior spaces 2b within the cathode-side block 2. Doing so enables the samples to be introduced and subjected to electrophoresis simultaneously in a plurality of short capillary tubes 1, enabling the analysis to be performed more quickly and with higher throughput.

Moreover, in the present embodiment, after a sample has been drawn into the interior space 2b of the cathode-side block 2 through a sample suction tube 4, the sample suction tube 4 is dipped into the buffer solution within the cathode-side reservoir 6, and the sample is introduced easily into the cathode-side end portion of the capillary tube 1 through merely applying a voltage to the cathode 7 within the cathode-side reservoir 6.

The anode-side end portion of the capillary tube 1 is always in a state that is in contact with the buffer solution that is held in the buffer solution retaining portion 31 of the anode-side reservoir block 3. As a result, when introducing the samples into the capillary tube 1 or when carrying out electrophoresis, all that is necessary is to apply the voltage to the anode 8 that is immersed in the buffer solution within the anode-side reservoir block 3, and there is no need to perform a separate operation for dipping the anode-side end portions of the capillary tubes 1 into the buffer solution.

### <Electrophoresis and Detection>

A voltage is applied between the cathode 7 and the anode 8 to perform electrophoretic separation on the samples that have been introduced into the individual capillary tubes 1. In this case, the application of the voltage between the cathode 7 and the anode 8 with an electric field strength of 230 V/cm, for example, as the voltage for separation, enables electrophoresis to be carried out well on the samples within the individual capillary tubes 1. The DNA fragments that are separated through electrophoresis are subjected to fluorescent detection in the detecting portion 21.

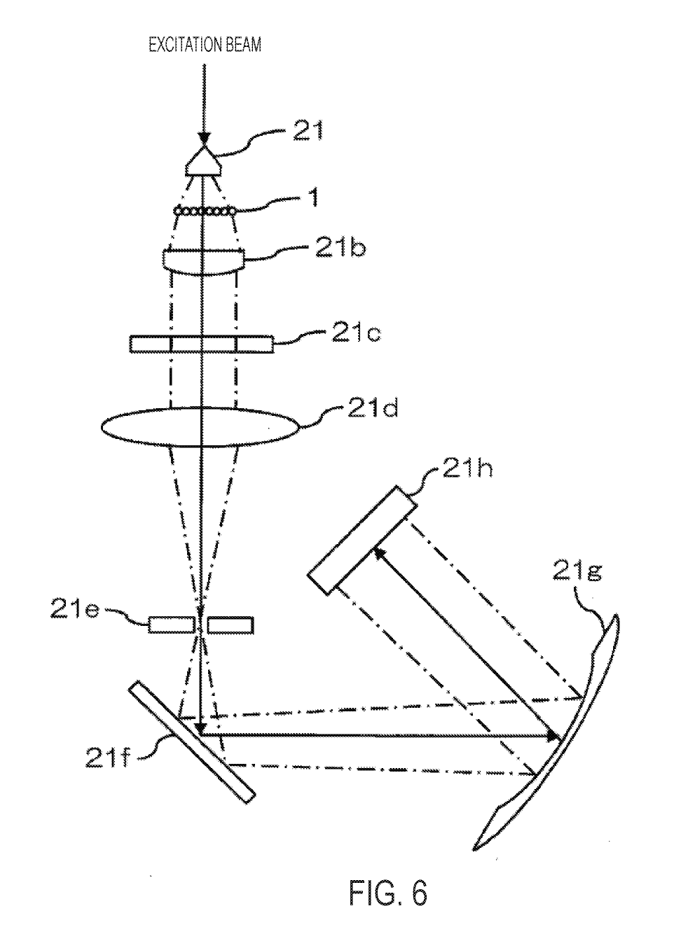

FIG. 6 is a diagram illustrating an example configuration for the detecting portion 21. In this example, a laser beam with a wavelength of 505 nm is used as an excitation beam, where each capillary tube 1 is illuminated with a uniform excitation beam from the detecting window 1c through a Powell lens 21a. The light (fluorescence) from each capillary tube 1 is collimated by a collimating lens 21b, and passes through a filter 21c to be incident on a focusing lens 21d.

Light that is focused by the focusing lens 21d passes through a slit that is formed in a slitted plate 21e, to be incident onto a diffraction grating 21g after reflecting off of a mirror 21f. The diffraction grating 21g is, for example, a reflective toroidal diffraction grating, and the light that is incident onto the diffraction grating 21g is spectroscopically split, through wavelength dispersion and through spatial dispersion of the capillary tubes 1, and detected by a photodetecting portion 21h. The photodetecting portion 21h may be structured from, for example, an area scan CMOS image sensor. The signal (fluorescence signal) outputted by the photodetecting portion 21h is sent to a personal computer, where data analysis is performed.

### <Completion of the Analysis, and Rinsing>

After the completion of analysis, the cathode-side block 2 and the capillary tube was 1 are rinsed automatically through the procedure set forth below. First, in a state wherein the pipe 51 and the pipe 53 have been connected by the electromagnetic switching valve 16, the individual sample suction tubes 4 are dipped by the auto-sampler 19 into the rinsing water of the drain 18, and the pump 5 is driven. Through this, the rinsing water within the drain 18 is fed to the pump 5 side through each of the sample suction tubes 4 and the interior spaces 2b of the cathode-side block 2, to be drained to the outside through the opening/closing valve 17, which is in the open state.

Thereafter, the polymer filling needle 9 is pressed, by the driving mechanism 15, into the connecting port 3c of the anode-side reservoir block 3, and the separating polymer is supplied to the polymer filling needle 9 from the polymer cartridge 10, to fill the separating polymer into each of the capillary tubes 1 from the anode-side end portions through the interior spaces 3b of the anode-side reservoir block 3. In this case, the separating polymer that flows out from the cathode-side end portions of each of the capillary tubes 1 is fed to the pump 5 side through the interior spaces 2b together with the rinsing water that is drawn in from each of the sample suction tubes 4, to be discharged to the outside through the opening/closing valve 17, which is in the open state.

After the separating polymer has been filled into each of the capillary tubes 1 for the prescribed time, the polymer filling needle 9 is removed from the connecting port 3c of the anode-side reservoir block 3 by the driving mechanism 15, and moved to the next sample introducing procedure. When the sequence of analysis programs has been completed, then the rinsing water supplying needle 11 is immersed into the rinsing water within the rinsing port 12 by the driving mechanism 15, and, in a state wherein the pipe 52 and the pipe 53 have been connected by the electromagnetic switching valve 16, the pump 5 is driven to draw the rinsing water into the pump 5 side.

Thereafter, the rinsing water supplying needle 11 is pressed by the driving mechanism 15 into the connecting port 3c of the anode-side reservoir block 3, and the pump 5 is driven to supply rinsing water from the anode-side end portion to each of the capillary tubes 1 through the interior spaces 3b of the anode-side reservoir block. The rinsing water that is supplied into each of the capillary tubes 1 is guided from the cathode-side end portions through the cathode-side block 2 to each of the sample suction tubes 4, to drain into the drain 18 from each of the sample suction tubes 4.

The buffer solution within the buffer solution retaining portion 31 of the anode-side reservoir block 3, after being drawn in by the rinsing water supplying needle 11, is drained into the rinsing port 12, where the outside surface of the rinsing water supplying needle 11 is also rinsed within the rinsing port 12. Moreover, the polymer filling needle 9 is also rinsed within the rinsing port 12.

In the present embodiment, the insides of the capillary tubes 1 can be rinsed easily through merely supplying rinsing water from the anode-side end portions of the capillary tubes 1 through the interior spaces 3b of the anode-side reservoir block 3. Moreover, the interior spaces 3b of the anode-side reservoir block 3, and the buffer solution retaining portion 31 that is connected to the interior spaces 3b, can also be rinsed with the rinsing water, and because, after rinsing, the anode-side end portions of the capillary tubes 1 will be in a state wherein they are in contact with the rinsing water, this can prevent the anode-side end portions from drying out. Moreover, this process can be performed automatically, enabling an improvement in maintainability.

Moreover, in the present embodiment the tip end of the rinsing water supplying needle 11 can be inserted into the connecting port 3c of the anode-side reservoir block 3, and the rinsing water can be supplied to the capillary tubes 1 through the interior spaces 3b of the anode-side reservoir block 3 through the tip end of the rinsing water supplying needle 11. This makes it easy to force the rinsing water to flow into the capillary tubes 1.

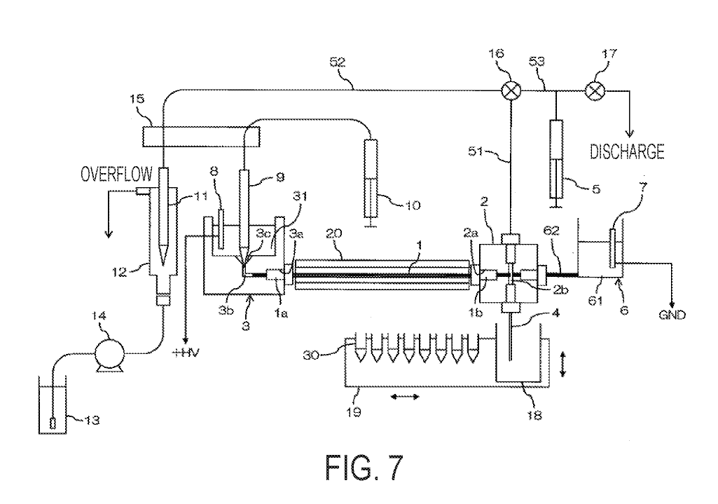

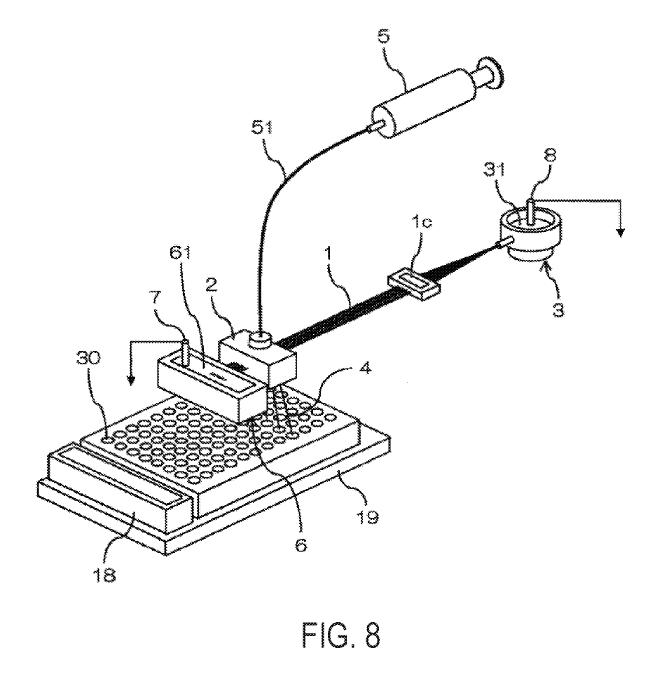

FIG. 7 is a schematic diagram illustrating an example configuration of a capillary electrophoresis apparatus according to another embodiment according to the present invention. Moreover, FIG. 8 is a perspective diagram illustrating a form wherein samples are introduced into each of the individual capillary tubes 1 in the capillary electrophoresis apparatus of FIG. 7. In the present embodiment, only the structures of the cathode-side reservoir 6 and the cathode-side block 2 differ from those in the embodiment set forth above, where the other structures are identical to those of the embodiment set forth above, and thus identical reference symbols are assigned to identical structures, and detailed explanations thereof are omitted.

The cathode-side reservoir 6 in the present embodiment is not structured on the auto-sampler 19, but instead is structured connected to the cathode-side block 2. Specifically, the buffer solution retaining portion 61 of the cathode-side reservoir 6 is connected to each of the interior spaces 2b of the cathode-side block 2 through individual connecting tubes 62 corresponding to the capillary tubes 1. Each of the connecting tubes 62 is connected to the cathode-side block 2 with respective interior spaces 2b on lines extending from the respective capillary tubes 1.

In this case, when the sample is drawn into an interior space 2b of the cathode-side block 2 through a sample suction tube 4, the voltage is applied to the cathode 7 within the cathode-side reservoir 6 after sucking also the buffer solution within the buffer solution retaining portion 61 of the cathode-side reservoir 6. This enables the samples to be introduced into each of the capillary tubes 1 from the cathode-side end portions, and enables the time required for introducing the samples to be shortened, thereby enabling the analysis to be carried out more quickly and with higher throughput.

The field to which the present invention can be applied includes a field known as “SBT” (Sequencing Based Typing) that performs genetic analysis through high-precision sequencing of relatively short base sequences through amplifying, through the PCR (Polymerase Chain Reaction) method only target genes on the genome and the regions to be analyzed for genetic mutations. As examples of individualized medical treatments wherein treatments and drug administration regimens are established through examining cancerous genetic mutations that are targets of molecule-targeted drugs there are the K-ras genes (exon2, and codon12 and 13) for colon cancer, the BRAF genes (exon15, and V600E), the EGFR genes (exon18, 19, and 21) for lung cancer, the c-kit genes (exon9 and 11) for gastrointestinal stromal tumors, and the like. These all can be examined with the sequencing length of the capillary electrophoresis apparatus according to the present invention.

### EXPLANATIONS OF REFERENCE SYMBOLS

1: Capillary Tube
2: Cathode-Side Block
2b: Interior Space
3: Anode-Side Reservoir Block
3b: Interior Space
3c: Connecting Port
4: Sample Suction Tube
5: Pump
6: Cathode-Side Reservoir
7: Cathode Electrode
8: Anode Electrode
9: Polymer Filling Needle
10: Polymer Cartridge
11: Rinsing Water Supplying Needle
12: Rinsing Port
13: Rinsing Water Tank
14: Rinsing Water Pump
15: Driving Mechanism
16: Electromagnetic Switching Valve
17: Opening/Closing Valve
18: Drain
19: Auto-Sampler
20: Temperature Controlling Portion
21: Detecting Portion
30: Sample Container
31: Buffer Solution Retaining Portion
48: Electromagnetic Switching Valve
51-53: Pipes
61: Buffer Solution Retaining Portion
62: Connecting Tube

What is claimed:

1. A capillary electrophoresis apparatus comprising:
a sample suction tube for drawing in a sample;
a pump that is driven in order to draw in a sample into the sample suction tube; and
a capillary tube, disposed straight in the horizontal direction, into which the sample that is drawn into the sample suction tube is introduced.

2. A capillary electrophoresis apparatus as set forth in claim 1, further comprising: a cathode-side block wherein a cathode-side end portion of the capillary tube, the sample suction tube, and the pump are connected, and wherein an interior space is formed wherein the flows thereof are joined.

3. A capillary electrophoresis apparatus as set forth in claim 2, wherein: a plurality of capillary tubes and a plurality of the sample suction tubes, corresponding to the individual capillary tubes, are connected to the cathode-side block, and a plurality of interior spaces, corresponding to the individual capillary tubes, is formed in the cathode-side block.

4. A capillary electrophoresis apparatus as set forth in claim 2, further comprising:
a cathode-side reservoir having a buffer solution retaining portion able to hold a buffer solution; and
a cathode that is immersed in the buffer solution in the cathode-side reservoir, wherein:
after a sample has been drawn into an interior space of the cathode-side block through the sample suction tube, a voltage is applied to the cathode within the cathode-side reservoir, in a state wherein the sample suction tube is dipped in the buffer solution within the cathode-side reservoir, to introduce a sample from the cathode-side end portion into the capillary tube.

5. A capillary electrophoresis apparatus as set forth in claim 2, further comprising:
a cathode-side reservoir that has a buffer solution retaining portion able to hold a buffer solution, where the buffer solution retaining portion is connected to an interior space of the cathode-side block; and
a cathode that is immersed in the buffer solution in the cathode-side reservoir; wherein
when a sample is drawn into an interior space of the cathode-side block through the sample suction tube, a voltage is applied to the cathode within the cathode-side reservoir, after buffer solution within the cathode-side reservoir has also been drawn in, to introduce a sample into the capillary tube from the cathode-side end portion.

6. A capillary electrophoresis apparatus as set forth in claim 1, further comprising:
an anode-side reservoir block able to hold a buffer solution within a buffer solution retaining portion, and wherein an interior space is formed connecting with an anode-side end portion of the capillary tube; and
a polymer filling mechanism for pressure-filling a separating polymer through the interior space of the anode-side reservoir block into the capillary tube from the anode-side end portion.

7. A capillary electrophoresis apparatus as set forth in claim 6, wherein:
the polymer filling mechanism includes a polymer filling needle that is inserted into the anode-side reservoir block; and
a connecting port, into which the tip end of the polymer filling needle can be inserted, and which can be sealed thereby, is formed at a boundary portion, in the anode-side reservoir block, between the buffer solution retaining portion and the interior space.

8. A capillary electrophoresis apparatus as set forth in claim 6, further comprising: a rinsing water supplying mechanism for supplying rinsing water through the interior space of the anode-side reservoir block to the capillary tube from the anode-side end portion.

9. A capillary electrophoresis apparatus as set forth in claim 8, wherein: the rinsing water supplying mechanism includes a rinsing water supplying needle wherein the tip end thereof can be inserted into the connecting port.

10. A capillary electrophoresis apparatus as set forth in claim 1, further comprising:
a temperature controlling portion, provided straight along the direction in which the capillary tube extends, able to contain, and control the temperature of, the capillary tube therein; wherein:
the capillary tube can be removed to the outside of the temperature controlling portion through moving the temperature controlling portion in a direction that is perpendicular to the direction in which the capillary tube extends.
